(19) United States
(12) Patent Application Publication
(10) Pub. No.: US 2012/0109302 A1
(43) Pub. Date: May 3, 2012

(54) MEDICAL IMPLANT AND METHOD FOR PHOTODYNAMIC THERPY

(75) Inventors: Keith E. Miller, Germantown, TN (US); Bret Matthew Wilfong, Hernando, MS (US)

(73) Assignee: Warsaw Orthopedic, Warsaw, IN (US)

(21) Appl. No.: 12/913,504

(22) Filed: Oct. 27, 2010

(51) Int. Cl. A61F 2/44; B23P 11/02; A61B 17/56

(52) U.S. Cl. 623/17.11; 606/86 A; 29/446

(57) ABSTRACT

An implant and method for insertion between adjacent vertebral members. The implant comprises an implant body with a base section having a plurality of base extensions, and an end cap adapted for selective axial positioning at a selected point on the base section. The end cap comprises a locking apertures and positioning apertures configured to engage corresponding base extensions, and a variable aperture slot extending through the end cap which is adapted to enable the end cap to resiliently deform as the end cap is positioned on the base section. The locking apertures and positioning apertures will prevent rotational movement of the end cap relative to the base section once the end cap is secured to the base section. The implant imparts end cap height and angulation to an adjacent vertebral body at the selected or desired point when the implant is positioned in the intervertebral space.

### BACKGROUND

The present application is directed to implants, devices and methods for stabilizing vertebral members, and more particularly, to intervertebral implants, devices and methods of use in replacing an intervertebral disc, a vertebral member, or a combination of both to distract and/or stabilize the spine.

The spine is divided into four regions comprising the cervical, thoracic, lumbar, and sacrococcygeal regions. The cervical region includes the top seven vertebral members identified as C1-C7. The thoracic region includes the next twelve vertebral members identified as T1-T12. The lumbar region includes five vertebral members L1-L5. The sacrococcygeal region includes nine fused vertebral members that form the sacrum and the coccyx. The vertebral members of the spine are aligned in a curved configuration that includes a cervical curve, thoracic curve, and lumbosacral curve. Intervertebral discs are positioned between the vertebral members and permit flexion, extension, lateral bending, and rotation.

Various conditions and ailments may lead to damage of the spine, intervertebral discs and/or the vertebral members. The damage may result from a variety of causes including, but not limited to, events such as trauma, a degenerative condition, a tumor, or infection. Damage to the intervertebral discs and vertebral members can lead to pain, neurological deficit, and/or loss of motion of the spinal elements.

Various procedures include replacing a section of or the entire vertebral member, a section of or the entire intervertebral disc, or both. One or more replacement implants may be inserted to replace the damaged vertebral members and/or discs. The implants are configured to be inserted into the intervertebral space and contact against adjacent vertebral members. The implants are intended to reduce or eliminate the pain and neurological deficit, and increase the range of motion.

The curvature of the spine and general shapes of the vertebral members may make it difficult for the implants to adequately contact the adjacent vertebral members or to position the adjacent vertebral members in a desired orientation. There is a need for implants or devices configurable to match the spinal anatomy for secure contact and/or desired orientation for secure contact when implanted into an intervertebral space.

### SUMMARY

The present application discloses implants or devices for insertion into an intervertebral space between a first and second vertebral member. The implant imparts an end cap angulation to an adjacent vertebral body at a selected point when the implant is positioned in the intervertebral space. The implant comprises an implant body having base section with a plurality of base extensions and an end cap adapted for selective positioning at the selected point on the base section. The end cap comprises an exterior contact surface that faces away from the implant body when the end cap is positioned on the base section, a seating surface adapted to contact the base section when the end cap is positioned on the implant body, an end cap angulation, at least two locking apertures adapted to receive and engage corresponding base extensions, at least one positioning aperture adapted to receive a base extension, and a variable aperture slot which enables the end cap to resiliently deform as the end cap is positioned on the base section. The locking apertures and positioning apertures are configured to complementarily engage corresponding base extensions to securely maintain the end cap positioned on the base section. The locking apertures and positioning aperture prevent rotational movement of the end cap relative to the base section. The end cap angulation comprises an angular value in the range of between zero degrees to fifteen degrees (0°-15°).

The present application also discloses an end cap for use with an implant having a base section with at a plurality of base extensions. The end cap is axially placed on the base section positioned or moved into a secure or locked position on the base section. The end cap positioned on the implant imparts an end cap angulation to an adjacent vertebral body at a selected point when the implant is positioned in an intervertebral space. The end cap comprises an exterior contact surface, a seating surface, a substantially vertical exterior cap wall extending between the exterior contact surface and the seating surface, at least two locking apertures extending between the exterior contact surface and the seating surface and adapted to receive and engage corresponding base extensions, at least one positioning aperture extending between the exterior contact surface and the seating surface and adapted to receive and engage a base extension, and a variable aperture slot extending between the exterior contact surface and the seating surface and adapted to enable the end cap to resiliently deform as the end cap is positioned on the base section. The locking apertures and positioning apertures are configured to simultaneously and complementarily engage corresponding base extensions to securely maintain the end cap positioned on the base section, and to enable the end cap to be axially positioned at a selected point on the implant base section. The end cap angulation comprises an angular value in the range of between zero degrees to fifteen degrees (0°-15°).

There is also provided a method of assembling an implant for insertion into an intervertebral space between a first and second vertebral member. The method comprises positioning an end cap at an end of an implant body, the end cap having a fixed aperture adapted to receive a corresponding first base extension that extends from the implant body, a variable aperture adapted to receive a corresponding second base extension that extends from the implant body, and a least one position securing apertures adapted to receive corresponding base extensions that extend from the implant body; axially inserting the first and second base extensions into a corresponding end cap fixed aperture and variable aperture, and at least one base extension into the at least one position securing apertures; resiliently deforming the end cap via a variable aperture slot during axial insertion of the end cap onto the implant body to enable the first, second and third locking protrusions to lockingly engage the first and second base extensions in the fixed and variable apertures, and to permit base extensions to be received in corresponding position securing apertures; and securing the first and second base extensions within the corresponding fixed and variable apertures, and positioning the base extensions into the at least one position securing apertures to thereby position the end cap to the implant body in a locked position.

The various aspects of the various embodiments may be used alone or in any combination, as is desired.

### BRIEF DESCRIPTION OF THE DRAWINGS

FIG. 1 is a schematic diagram of an implant according to one embodiment positioned in an intervertebral space between vertebral members;

FIG. 2 is a perspective view of an implant with an end cap attached thereon according to one embodiment;

FIG. 3 is an exploded perspective view of the implant and end cap of FIG. 2;

FIG. 4 is a top view of the implant and end cap of FIG. 2;

FIG. 5 is a section view along the line A-A of the implant and end cap of FIG. 4;

FIG. 6 is a front perspective view of an implant end cap according to one embodiment;

FIG. 7 is a rear perspective view of the implant end cap of FIG. 6;

FIG. 8 is a top view of the end cap of FIG. 6;

FIG. 9 is a top view of an implant end cap according to a second embodiment;

FIG. 10 is a top view of an implant end cap according to a third embodiment; and

FIG. 11 is a top view of an implant end cap according to a fourth embodiment.

### DETAILED DESCRIPTION

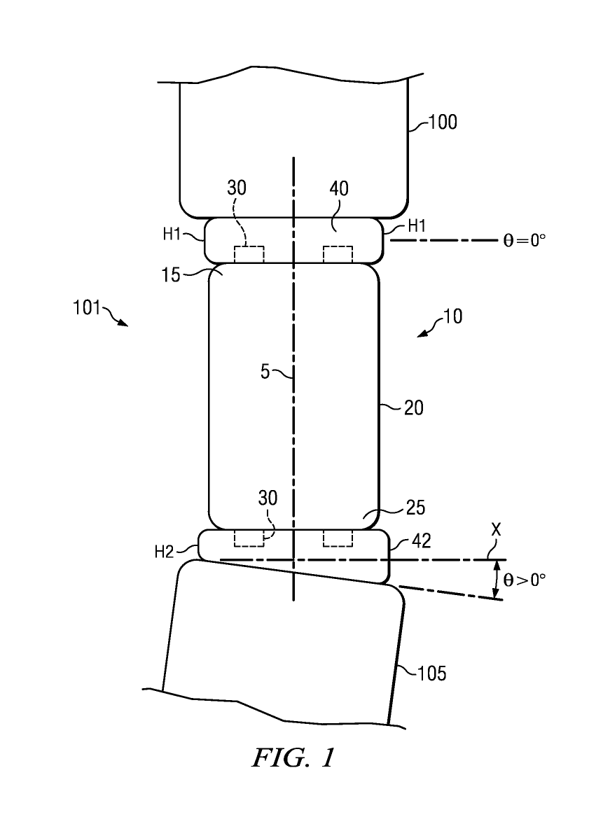

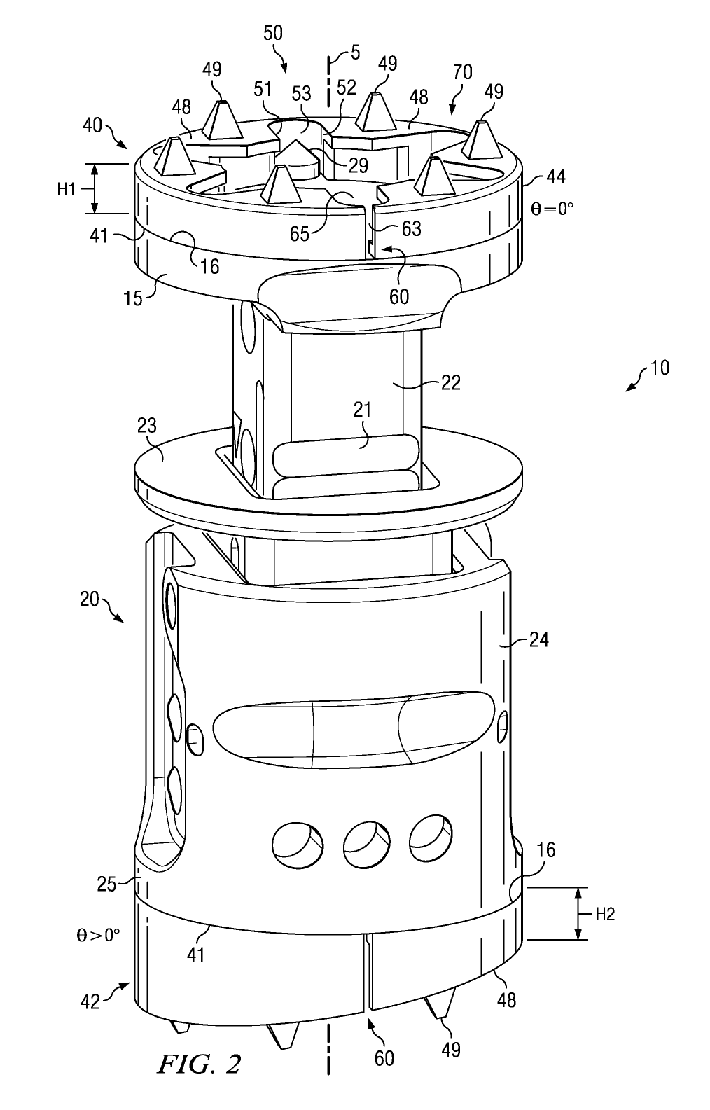

The present application is directed to intervertebral implants for spacing apart vertebral members 100 and 105. FIG. 1 shows an implant 10 positioned within an intervertebral space 101 formed between vertebral members 100 and 105. The implant 10 includes an implant body 20 and one or more end caps 40 and 42 which are attached to the implant body 20 at a first or second implant base section 15 and 25 via base teeth or base extensions 30. The end caps can be an upper end cap 40 or a lower end cap 42. The one or more end caps 40 and 42 will attach or connect to the implant body 20 to impart a desired or selected angulation θ, an angular orientation and/or an end cap position to the adjacent vertebral member 100 or 105. A securing mechanism comprised of a first and second locking mechanism 50 and 60, shown in one aspect in FIGS. 2, 4 and 5, which engage and lock the end cap 40 and 42 to the implant base section 15 and 25. The securing mechanism further comprises a third position securing aspect or feature 70, shown in FIGS. 2, 4 and 5-8, which engage the end cap 40 and 42 to the implant base section 15 and 25 to substantially prevent rotational movement of the end cap 40 or 42 relative to the implant base section 15 or 25. The end caps 40 or 42 disclosed herein will improve the contact and stability of the intervertebral implant 10 to the adjacent vertebral members 100 and 105 and drive angular orientation and position for correction and/or improved alignment of the spine.

As shown in FIG. 1, the implant 10 may include first and second end caps 40 and 42 positioned at opposite ends of the implant body 20 at first and second base sections 15 and 25. The first end cap may be an upper end cap 40 and the second end cap may be a lower end cap 42. A first end cap 40 can have an angulation θ of zero degrees and a first end cap height H1, as shown in FIGS. 1-3, and 5-7. The second end cap 42 can have an angulation θ greater than zero degrees, for example of 15° degrees, and a second height H2. Those of skill in the art will recognize that the first and second end caps 40 or 42 may have the same or different configuration, heights H, and/or the same or different end cap angulation θ. Further, although two end caps 40 and 42 are shown in the disclosed aspects, those of skill in the art will recognize that one or two end caps 40 or 42 may instead be used in a medical procedure with the implant 10, and that the end caps can be attached to either the first and second base sections 15 and 25, to impart desired or needed heights H and angulation θ to adjacent vertebral members 100 or 105 to thereby correct, improve and/or stabilize the affected spinal anatomy.

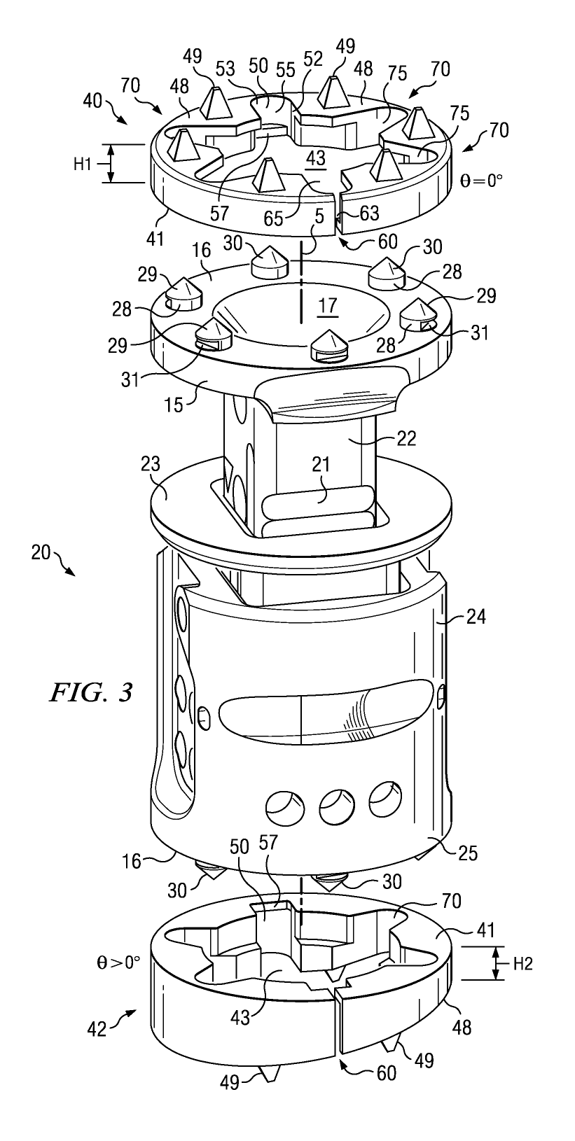

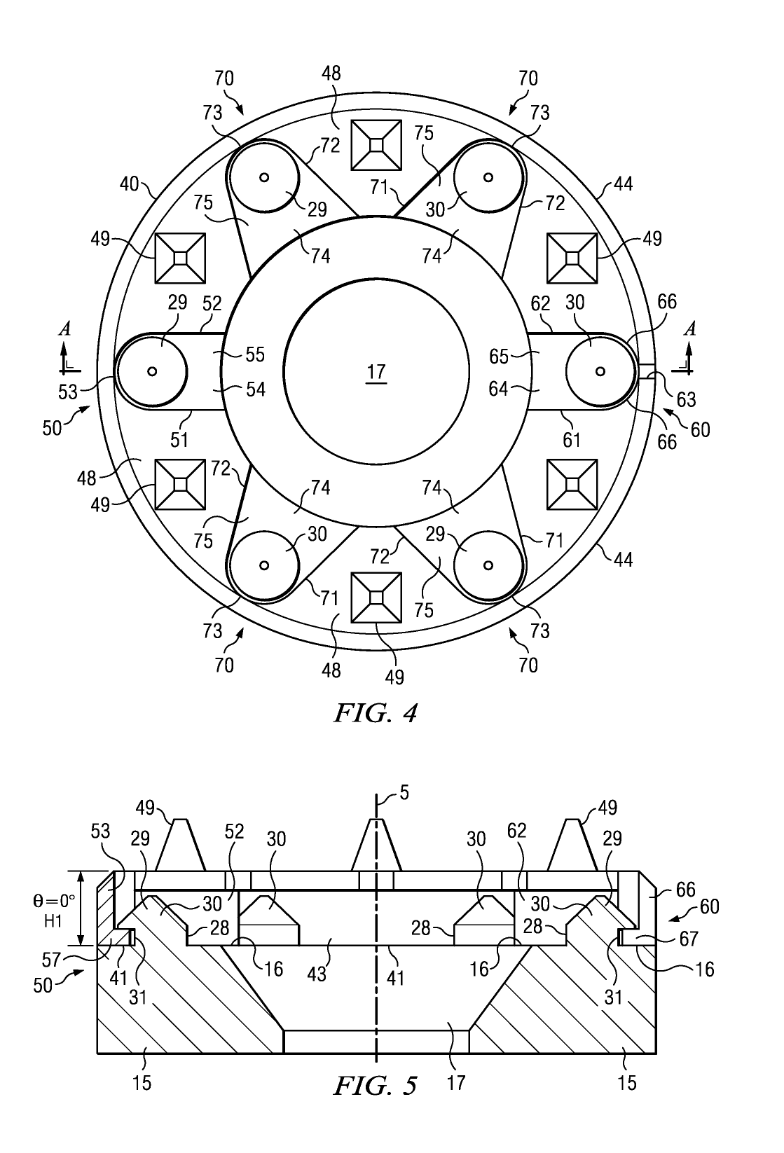

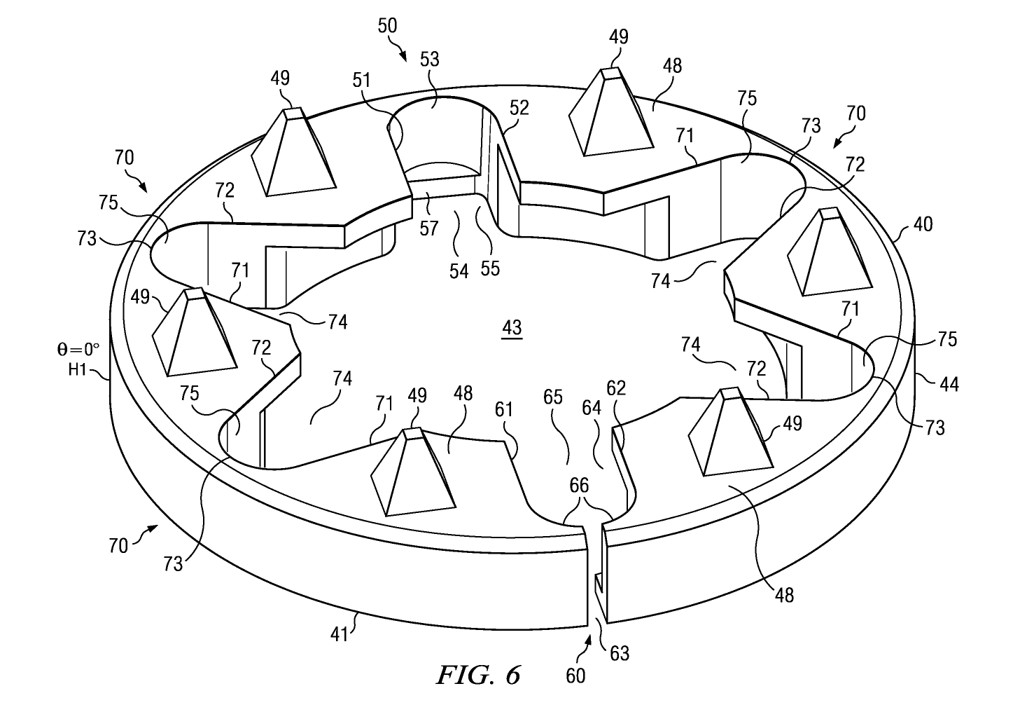

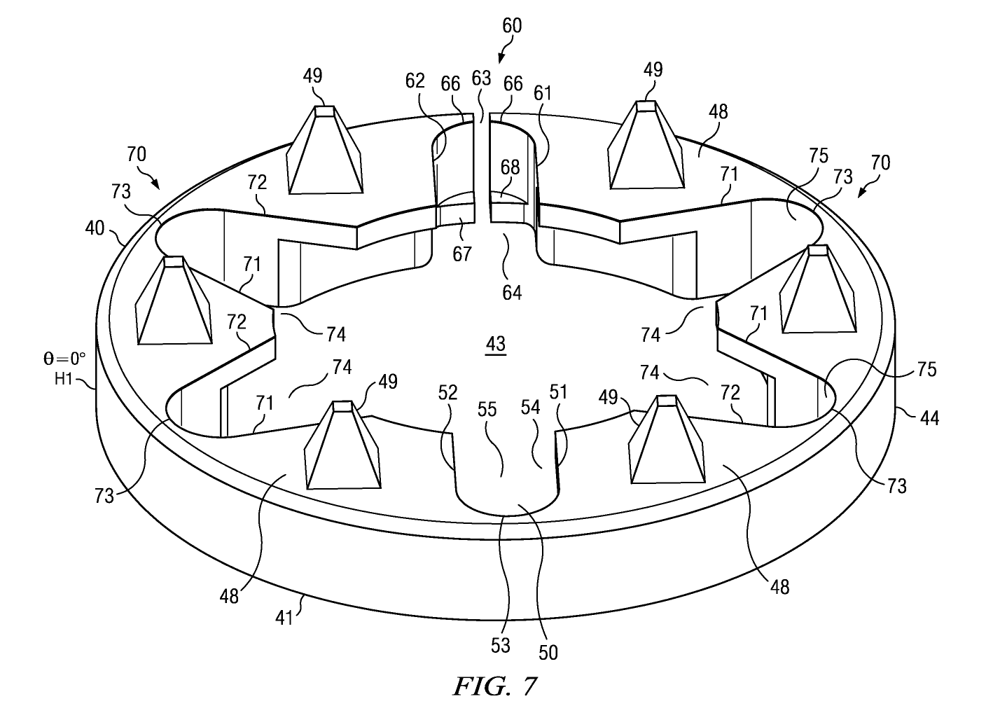

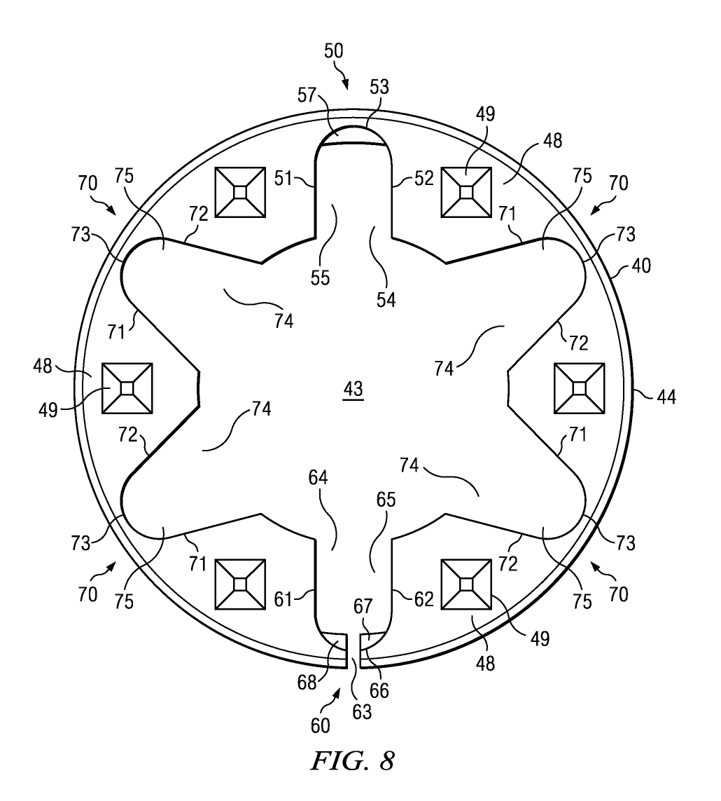

FIGS. 2-3 illustrate assembled and exploded perspective views of an implant 10 with upper and lower end caps 40 and 42 according to one embodiment. FIG. 4 is a top view of the assembled implant 10 and end cap 40 of FIG. 2. FIG. 5 is section view along the line A-A of the assembled implant 10 and end cap 40 shown in FIG. 4 showing in more detail the securing or locking mechanism with a first securing feature or locking mechanism 50, second securing feature or locking mechanism 60 and third securing position feature 70. FIGS. 6 and 7 are perspective views of the end cap 40, and FIG. 8 is a top view of the end cap 40, illustrating in more detail the first securing or locking mechanism 50 with a fixed locking aperture 55, the second securing or locking mechanism 60 with a variable locking aperture 65 and flexing aperture slot 63, and the third securing feature 70 with a position securing aperture 75.

The implant body 20 in combination with the end caps 40 and 42 is sized to fit within the intervertebral space 101. In this aspect, the implant body 20 is constructed of two implant sections 22 and 24 which are movable relative to each other to permit axial adjustment of the overall axial height of the implant 10. The implant body 20 includes an inner implant body 22 adapted to axially travel inside an outer implant body 24 to thereby enable selected or controlled collapse and expansion of the implant 10. The inner implant body 22 includes a first base section 15 and the outer implant body 24 includes a hollow interior and a second base section 25. The inner implant body 22 is sized to fit within and axially travel along the hollow interior of the outer implant body 24 to adjust the height of the implant body 20 along the longitudinal axis 5. The inner body 22 includes a neck area with a plurality of scallops 21 that extend along the length of the inner body 22. Both the inner and outer implant bodies 22 and 24 may be hollow and include one or more apertures to receive bone growth material. Also, one or more apertures may extend through the body 20 walls to the hollow interior. The implant body 20 may also be constructed from a single section with a fixed height measured between the first and second base sections 15 and 25.

A securing mechanism 23 may secure the inner and outer sections 22 and 24 together to fix the height. In one embodiment, the securing mechanism 23 is configured to receive one or more cylindrical rods (not illustrated) that seat within the plurality of scallops 21 that extend along the inner body 22 neck. U.S. Patent Publication No. 2008/0114467 discloses embodiments of an implant that may be used with end caps and include a multiple-section body and a locking mechanism and is herein incorporate by reference in its entirety.

FIGS. 2-5 illustrate the implant body 20 which is configured to receive an end cap 40 or 42 at the first and second base sections 15 and 25. The first and second base sections 15 and 25 have an exterior support surface or support surface 16 that extends around the periphery of a corresponding central base aperture 17. In the disclosed embodiment, the support surface 16 is substantially flat, although other embodiments may include a variety of different surface configurations.

FIGS. 3-5 also show that the base sections 15 and 25 include a plurality of base teeth or base extensions 30 that extend axially away or outward from the support surface 16. The base extensions 30 are preferably evenly spaced around the periphery of the base section 15 and 25 and extend away from an exterior surface 16 of the first base section 15 in a substantially outward or axial direction. The base extensions 30 are sized and configured to extend into corresponding end cap locking or securing apertures or passages 55, 65 and 75 when the end cap 40 or 42 is positioned onto the base section 15 and 25. The outer implant body 24 includes a second base section 25 with similarly positioned and configured base teeth 30. FIGS. 3 and 4 illustrate an embodiment where the implant base sections 40 or 42 have six (6) extending base teeth or base extensions 30 that are equidistantly spaced about the support surface 16 about or around the periphery of the central base aperture 17. As such, in this embodiment, the six extending base teeth 30 are spaced at or about 60° apart from each other around the periphery of the implant base section 15 or 25.

Other base section 15 or 25 embodiments are contemplated which could include at least two or more than two base extensions 30. In such a case, there must be at least the same number of locking or securing apertures or passages 55, 65 and 75 to accept the base extensions 30. There could also be more securing apertures or passages 55, 65 and 75 than base teeth 30 which will result in greater degree of control in axial placement of the end cap 40 or 42 on the base section 15 or 25. Further, other contemplated embodiments include base extensions 30 which are non-equidistantly spaced about the support surface 16 about or around the periphery of the central base aperture 17. In such cases, the locking or securing apertures or passages 55, 65 and 75 will be spaced or located at corresponding non-equidistant points around the end cap area between the vertical exterior cap wall 44 and the central aperture 43 to accept the base teeth 30. In the non-equidistant spacing case, the locking or securing apertures 55, 65 and 75 are preferably spaced and located to complementarily align with the spacing and location of the base teeth 30 to permit axial end cap 40 insertion and rotational engagement with the base teeth 30 of the implant base section 15 or 25. The implant base section's 15 and 25 central base aperture 17, shown in FIGS. 2, 3 and 5, is adapted to receive or permit delivery of bone growth material into the implant 10 which will augment fusion in the disc space 101 once the implant is in place between the vertebral members 100 and 105. The base apertures 17 are preferably adjacent and aligned with a corresponding end cap central aperture 43. Those of skill in the art will recognize that the base aperture 17 and end cap central apertures 43 may also be non-aligned if desired or needed by a surgeon, medical procedure or clinical application.

In the aspect shown in FIGS. 3-5, the base teeth or base extensions 30 include a tooth base or stem 28 that extends axially outward from the support surface 16 and which is capped with a tooth head 29. The tooth head 29 includes a tapered shape or configuration, for example similar to a solid cone shape that terminates at a tip. The tooth head tip facilitates entry and travel into a corresponding end cap locking or securing aperture or passage 55, 65 and 75 when the end cap 40 or 42 is positioned and placed on the base section 15 and 25. The tooth head tip may also be appropriately shaped to directly contact against and/or penetrate into an adjacent vertebral member 100 or 105 when the implant 10 is used without an end cap 40, or when the base tooth or base extension 30 extends beyond the end cap 40. As best illustrated in FIGS. 3 and 5, the tooth base or stem 28 comprises a smaller width than the tooth head 29 forming an undercut tooth section or notch 31. The depth of the undercut tooth section 31 may be the same or different for each of the base teeth or base extensions 30. The undercut tooth sections 31 may face radially outward from the central base aperture 17. One or more base teeth or base extensions 30 may include a tooth base or stem 28 that has substantially the same width as the tooth head 29. In some embodiments, the width of the tooth stem 28 is equal to the widest part of the tooth head 29.

The end cap 40 or 42, via its end cap locking apertures or passages 55, 65 and 75, can be attached to the implant body 20 via the base teeth or base extensions 30 when the end cap 40 or 42 is placed and positioned on the base section 15 and 25. The overall width or distance across the end cap 40 or 42 preferably matches the width or distance across the base section 15 and 25 such that the end cap 40 or 42 does not extend past the lateral side walls of the implant body 20. In other embodiments, the end cap 40 or 42 may have a width or distance across the end cap 40 or 42 that is greater or smaller than width or distance across the base section 15 and 25 such that the end cap 40 or 42 would extend or would not extend, respectively, past the lateral side walls of the implant body 20 depending on the desire or needed of a surgeon, medical procedure or clinical application.

FIGS. 2-4 and 6-8 illustrate an end cap 40 which comprises an annular or circular like shape with an outside or exterior contact surface 48, locking or securing apertures or passages 55, 65 and 75, an interior or seating surface 41, a substantially vertical exterior end cap wall 44 and a central aperture 43. The end cap 40 may take on a variety of geometric shapes as may be desired or needed by a surgeon, medical procedure or clinical application. The end cap could also take on other shapes including, but are not limited to, polygonal and crescent-shaped. The end cap 40 may also include a central aperture 43 that may also have various geometric shapes.

The exterior contact surface 48 and the seating surface 41 are bounded by the vertical exterior end cap wall 44 and the central aperture 43. The seating surface 41 is preferably substantially flat to complementarily abut against the exterior support surface 16 of the implant base section 15 or 25. In a preferred aspect, the seating surface 41 and the exterior surface 16 have complementary and substantially flat surfaces such that the end cap 40 can seat flush on the implant base section 15 when placed in an engaged and locking position. The exterior contact surface 48 extends around the central aperture 43. The exterior contact surface 48 may be flat, or may include various other configurations to facilitate contact with the vertebral member 100 or 105. Those of skill in the art will recognize that the seating surface 41 and exterior contact surface 48 may take on other configurations as may be desired or needed by a surgeon, medical procedure or clinical application. The central aperture 43 is preferably aligned with and the same size as the corresponding base aperture 17. The central aperture 43 and base aperture 17 may also be of different sizes and non-aligned if desired or needed by a surgeon, medical procedure or clinical application.

The exterior contact surface 48 includes end cap teeth 49 which will engage the end plates of an adjacent vertebral member 100 or 105 to assist the implant 10 grip the vertebral member end plate, provide implant 10 stability in the disc space 101, and prevent implant 10 ejection from the intervertebral space 101. The end cap teeth or spikes 49 may be a series of equidistantly spaced end cap teeth or spikes 49 extending from the end cap exterior surface 48, as shown in FIGS. 2-8. Those of skill in the art will recognize that the number, size, shape, orientation and spacing of the end cap teeth 49 may vary according to the needs of a medical procedure, clinical application, or surgeon need or selection. For example, the end cap teeth or spikes 49 could also be a series or pattern of uniform knurls and spikes 49 (not shown) that cover the end cap exterior surface 48 and assist in providing a securing and stabilizing function of the combined end cap 40 or 42 and implant body 20 or solely a series or pattern of uniform knurls (not shown) that cover the end cap exterior surface 48, so long as they assist in providing a securing and stabilizing function of the combined end cap 40 or 42 and implant body 20. Those of skill in the art will recognize that the number, size, height, shape, orientation and spacing of the end cap teeth or spikes 49 may vary according to the needs of a medical procedure or clinical application.

The end cap teeth 49 may contact the adjacent vertebral member 100 or 105 and/or penetrate into the vertebral member 100 or 105 as may be desired or required by a physician or medical procedure or clinical application. In one aspect, the end cap teeth or spikes 49 will come in contact with and engage the end plates of an adjacent vertebral body 100 or 105 once the combined implant body 10 and end cap 40 or 42 is positioned in an intervertebral space 101 between the vertebral members 100 and 105. The end cap teeth or spikes 49 will extend from the end cap exterior surface 48 sufficiently to grip, penetrate and embed into the adjacent vertebral member 100 and 105 end plate to thereby provide implant stability in the intervertebral disc space 101 and prevent the inserted implant 10 from being ejected out of the intervertebral space 101 after implant 10 insertion. The end cap teeth or spikes 49 will provide a securing and stabilizing function of the combined end cap 40 and implant body 10. The actual height of the end cap teeth or spikes 49 can vary to accommodate the selection or need of a surgeon, medical procedure or clinical application. When an implant 10, with positioning base teeth 30 and one or two end caps 40 or 42, is inserted into an intervertebral space 101 and set to a desired implant height, via appropriate instruments (not shown), the protruding end cap teeth or spikes 49 will grip and/or penetrate into the adjacent vertebral member end plate to maintain a stable implant 10 position between the adjacent vertebral members 100 and 105.

The end cap 40 or 42 preferably further comprises an angulation aspect θ and an end cap vertex height H. The end cap angulation θ and cap height H may have a range of values as may be selected or needed by a surgeon, medical procedure or clinical application. In one aspect, preferred discrete values for end cap angulation are 0°, 4°, 8° and 15° degrees measured from an angulation reference line X, shown in FIG. 1. In other embodiments, the preferred angulation θ values may be in the range of zero and thirty degrees (0°-30°), with a preferred range of between zero and fifteen degrees (0°-15°). In one aspect, the cap height H may have preferred values in 1.0 mm or 0.5 mm increments measured from the end cap seating surface 41. The angulation reference line X is preferably at the cap height H value as shown in FIG. 1. The end cap's angulation θ is a measure of the inclination of the exterior contact surface 48 relative to the angulation reference line X. Insertion of an implant 10 with an end cap 40 or 42 having an angulation θ aspect enables the end cap 40 or 42 to impart a desired or selected angulation θ to an adjacent vertebral member 100 or 105. In this manner, selective angulation θ can be imparted to the adjacent vertebral body 100 or 105 and thereby assist in the correction and/or improved orientation, stabilization and alignment of the spine. In the event where additional implant height H is desired or required without any angulation, an end cap 40 having angulation θ of 0° degrees may be used to impart the additional height to the implant 10 in the amount of an end cap height H. Such a case is illustrated in FIGS. 2-3, and 5 which show views of an implant base section 15 with an end cap 40 having angulation θ of 0° degrees and a certain cap height H1. Additionally, selected angulation θ may advantageously and appropriately accommodate the lordotic or kyphotic shape of the spine depending upon the vertebral level at which the implant 10 is to be positioned in the patient.

FIGS. 1-3 show a first or upper end cap 40 and a second or lower end cap 42. The first and second end caps 40 or 42 may have the same or different configuration, heights H, and/or the same or different end cap angulation θ. As shown in FIGS. 1-3, the upper end cap 40 has an angulation θ of zero degrees and a first end cap height H1. FIGS. 1-3 show that the lower end cap 42 has an angulation θ greater than zero degrees (0°) and a second height H2. As noted previously, values for an end cap angulation can be 0°, 4°, 8° and 15° degrees measured from an angulation reference line X, or values in a range of zero and thirty degrees (0°-30°), with a preferred range of between zero and fifteen degrees (0°-15°). Those of skill in the art will recognize that end caps 40 or 42 with the same or different end cap angulation θ and the same or different end cap heights H1 or H2 may instead be used. Although two end caps 40 and 42 are shown in the disclosed aspects, those of skill in the art will also recognize that one end cap may instead be used, either as a lower or upper end cap, in a medical procedure with the implant 10 to impart desired or needed height H and angulation θ to adjacent vertebral members 100 or 105 and thereby correct, improve and/or stabilize the affected spinal anatomy.

FIGS. 1-3 show an aspect where the upper end cap 40 provides an angulation θ of zero degrees (0°) and is attached to the inner implant body 22 at the upper implant base section 15. In this aspect, the upper end cap 40 provides an end cap height H1 but will not provide any implant angulation θ. Such an end cap 40 may be used where there is a need only for additional height to augment the implant 10 in the amount of an end cap height H1 as might be desired or required by a surgeon, medical procedure or clinical application. FIGS. 1-3 also show an aspect where the lower end cap 42 provides an angulation θ greater than zero degrees (θ>0°) and attached to the outer implant body 24 at the lower implant base section 25. In this aspect, the lower end cap 42 provides an end cap height H2 and an implant angulation θ>0°. Such an end cap 42 may be used where there is a need for both additional height to augment the implant 10 in the amount of end cap height H2 and end cap angulation θ greater than zero degrees (θ>0°) as might be desired or required by a surgeon, medical procedure or clinical application.

FIGS. 2-4 and 6-8 show an end cap 40 which comprises one or more locking or securing apertures or passages 55, 65 and 75 that can receive corresponding base teeth 30 extending from the implant body 20 base sections 15 or 25. The locking or securing apertures 55, 65 and 75 are spaced around the end cap 40 or 42 to complementarily correspond to and accommodate the positioning of the base teeth or base extensions 30 extending from the base sections 15 or 25 when the end cap 40 or 42 is axially positioned or placed on the implant body 20.

In one aspect, shown in FIGS. 2-4 and 6-8, the fixed locking aperture 55 comprises a pair of substantially parallel sidewalls 51 and 52 bounded by an exterior curved wall 53 and an opposing interior open space 54. The fixed locking aperture 55 further includes a first locking protrusion or projection 57 which extends into the fixed locking aperture 55 from the exterior curved wall 53 between the sidewalls 51 and 52. As best shown in FIGS. 3, 5 and 6, the aperture protrusion or projection 57 extends into the fixed locking aperture 55 adjacent the seating surface 41 from the exterior curved wall 53. The fixed locking aperture or passage 55 is preferably sufficiently sized and configured to permit the base tooth head 29 to enter the end cap's fixed locking aperture 55 as the end cap 40 or 42 is axially mounted onto the implant body 20. The fixed locking aperture protrusion 57 is located adjacent the seating surface 41 and extends away from the exterior curved wall 53. This positioning locates the fixed aperture protrusion 57 such that it can fit under the tooth head 29 of the base tooth or base extension 30 and within the undercut section 31 when the end cap 40 or 42 is axially positioned and secured to the implant body 20, as best shown in FIG. 5.

The first locking protrusion or projection 57 interacts and cooperatively engages with the base tooth head 29 to enable the end cap 40 or 42 to be axially mounted and locked onto the implant body 20. The first locking protrusion or projection 57 in cooperation and interaction with the base tooth head's 29 underside and undercut section 31 permits the end cap 40 or 42 to be axially positioned and locked or secured to the implant body base sections 15 or 25. The first locking aperture protrusion 57 is configured to fit within the undercut section 31 and axially fit with the underside of the tooth head 29 to attach and secure the end cap 40 or 42 to the implant body 20.

When the end cap 40 or 42 is axially positioned on the base section 15 or 30, a base extension 30, via the tooth head 29, will interact with a corresponding fixed locking aperture protrusion 57 as the base extension 30 enters the fixed locking aperture 55. As the end cap 40 or 42 axially travels toward the fully seated position on the implant body base sections 15 or 25, the first locking aperture protrusion 57 slideably travels along the topside of the tooth head 29 towards the implant base section 15 or 25 and a secured or locking position. When the first locking aperture protrusion 57 travels past the edge of the tooth head 29 the first locking aperture protrusion 57 will move towards a secured or locking position under the tooth head 29 of the base tooth or base extension 30 and within the undercut section 31, as best shown in FIG. 5. Once the end cap 40 or 42 is axially and fully seated on the exterior surface 16 of the implant base section 15 or 25, the corresponding base extension 30 is located in the fixed locking aperture 55 in a secured or locking position.

Once the end cap 40 or 42 is seated in the fixed locking aperture 55, the combined first locking aperture protrusion 57 and underside of the tooth head 29 will obstruct and prevent axial travel or movement of the locked end cap 40 or 42 away from the implant body base sections 15 or 25. As will be discussed below, a variable aperture 65 and corresponding base tooth 30 interaction will simultaneously also obstruct and prevent axial travel or movement of the locked end cap 40 or 42 away from the implant body base sections 15 or 25. At this point, the first locking aperture protrusion 57 is in complementary and mechanical communication with the underside of the base tooth 29 and the undercut section 31 of the base tooth or base extension 30 and in an axially obstructive fit with underside of the base tooth 29 which locks or secures the positioning teeth 30 inside the fixed locking aperture 55 If an attempt is made to axially remove or impart axial travel on the end cap 40 or 42, the first locking aperture protrusion 57 will abut up against the underside of the tooth head 29 and prevent axial travel of the end cap 40 or 41 away from the implant body base sections 15 or 25. Further, if an attempt is made to rotationally move or force the end cap 40 or 42 to rotationally travel on the implant base section 15 or 25, the fixed locking aperture's 55 substantially parallel sidewalls 51 and 52 will prevent rotational movement or travel of the end cap 40 or 42 relative to the positioning teeth 30, as best shown in FIG. 4. The end cap 40 or 42 is now in a secured or locked position, shown in FIGS. 2 and 4, on the implant body base section 15 or 25. At this point, as shown in FIG. 5, the first locking aperture protrusion 57 is in complementary and mechanical communication with the underside of the tooth head 29 and the undercut section 31 of the base tooth or base extension 30 below the tooth head 29. The mechanical communication between the first locking aperture protrusion 57, the undercut section 31, the underside of the tooth head 29 and substantially parallel sidewalls 51 and 52 can comprise a first securing or locking mechanism 50.

In the secured or locked position, the end cap 40 or 42 is in an engaged or locked position relative to the implant body 20. The first locking aperture protrusion 57, the underside of the tooth head 29 and the undercut section 31 are preferably and complementarily configured such that, at the engaged and locked position, e.g., as shown in FIGS. 2, 4 and 5, the underside of the tooth head 29 prevents or interferes with axial movement of the first locking aperture protrusion 57 away from the base teeth 30, and thereby axial movement of the end cap 40 or 42 away from the positioning teeth 30 and base section 15 and 25.

The holding strength of the first locking mechanism 50 between the positioning teeth 30 and fixed locking aperture 55, via the first locking aperture protrusion 57, underside of the base tooth head 29, and the undercut section 31, may be augmented or controlled by the addition or use of a coating or adhesive substance between the first locking aperture protrusion 57, the base tooth head underside and the undercut section 31. For example, a coating, such a silicone, or an adhesives such as an epoxy, may be used to increase friction between the aperture protrusion 57, the base tooth head underside and the undercut section 31. Those of skill in the art will recognize that other substances or friction control mechanisms may be used to augment or control the holding strength between the fixed locking aperture 55 and the positioning teeth 30, such as roughened surfaces, dissimilar materials, and shape differences.

The complementary and mechanical communication between the first locking aperture protrusion 57, the base tooth head underside and the undercut section 31 will prevent axial movement or travel of the end cap 40 or 42 away from the implant base section 15 or 25 along the implant axis 5. This is the case since the first locking aperture protrusion 57 is now positioned underneath and obstructed by the base extension head 29. An attempt to axially move or remove the end cap 40 or 42 away from the implant base section would result in the first locking aperture protrusions 57 bumping into and abutting the underside of the teeth heads in the undercut sections 31. The tooth head 29 prevents axial movement of the end cap 40 or 42 away from the implant base section 15 or 25 along the implant axis 5 once the end cap 40 or 42 is in a secured or locked position with the base section 15 or 25. The number of fixed locking apertures 55 in an end cap 40 or 42 may vary from single to multiple fixed apertures 55. FIGS. 2-8 illustrate an embodiment with one (1) axially attaching fixed locking aperture 55. Once in a locked position, the fixed locking aperture 55 also prevents rotationally movement of the end cap 40 or 42 relative to the implant base section 15 or 25. This is accomplished via the fixed locking aperture's 55 substantially parallel sidewalls 51 and 52 into which the base teeth 30 will abut into if rotational movement or travel is attempted once in the locked position, as best shown in FIG. 4.

FIGS. 2-8 show that the end cap 40 or 42 further includes a variable locking aperture 65 that is adapted to receive a corresponding base tooth or base extension 30 extending from the implant body 20 base sections 15 or 25. In a preferred aspect, the variable locking aperture 65 will simultaneously operate in combination with the fixed locking aperture 55 to enable the end cap 40 or 42 to be axially attached and secured to the implant body 20 when the end cap 40 or 42 is axially placed on the base teeth 30 of the implant body base sections 15 or 25. FIGS. 2-8 show an embodiment with a single variable locking aperture 65 adapted to receive a corresponding base tooth or base extension 30 extending from the implant body 20 base sections 15 or 25 when the end cap 40 or 42 is axially attached on the implant body 20. FIGS. 2-8 show an embodiment of an end cap 40 or 42 with one fixed locking aperture 55 and one variable aperture 61. Those of skill in the art will recognize that an end cap 40 or 42 could have a varying combination of fixed and variable locking apertures 55 and 65. The number of fixed locking apertures 55 and variable locking apertures 65 may be same or a different as may be desired or needed by a surgeon, medical procedure or clinical application. For example, in another aspect, the end cap 40 or 42 could instead have two fixed apertures 55 and two variable locking apertures 65. Further, the fixed locking aperture 55 and variable locking aperture 65 may have the same or different shape, configuration and/or sizes so long as they are complimentarily configured to lockingly accept corresponding base teeth 30.

As best shown in, shown in FIGS. 2-8, the variable locking aperture 65 preferably comprises a pair of substantially parallel sidewalls 61 and 62 bounded by an opposing interior open space 64, and an exterior curved wall 66 comprising an aperture slot 63. The variable locking aperture 65 further includes a second and third locking protrusion or projection 67 and 68 which extend into the variable locking aperture 65 from the exterior curved wall 66 between the sidewalls 61 and 62. The second and third locking protrusions or projections 67 and 68 are separated by the variable aperture slot 63. In a preferred aspect, the second and third locking protrusions or projections 67 and 68 are mirror images of each other, as best shown in FIG. 8. However, those of skill in the art will recognize that the second and third locking protrusions or projections 67 and 68 may have the same or different configurations. Also, those of skill in the art will recognize that there may be one of more locking protrusions or projections 67 or 68 instead of two as might be desired or required by a surgeon, medical procedure or clinical application. FIGS. 2-8 illustrate a preferred embodiment with one (1) axially attaching or positioning variable locking aperture 65. However, the number of variable locking apertures 65 in an end cap 40 or 42 may vary from a single to multiple variable apertures 65.

The second and third locking protrusions or projections 67 and 68 of the variable locking aperture 65 interact and cooperatively engage with a corresponding base tooth head 29 to enable the end cap 40 or 42 to be axially mounted onto the implant body 20. The variable locking aperture or passage 65 is preferably sufficiently sized and configured to permit the base tooth head 29 to enter the end cap's variable locking aperture 65 as the end cap 40 or 42 is axially mounted or positioned onto the implant body 20 base section 15 or 25. The second and third locking protrusions or projections 67 and 68 in cooperation and interaction with the corresponding base tooth head's 29 underside and undercut section 31 permit the end cap 40 or 42 to be axially positioned and locked or secured to the implant body 20.

In one aspect, as best shown in FIGS. 3, 5 and 7, the second and third aperture protrusions or projections 67 and 68 extend into the variable locking aperture 65 adjacent the seating surface 41 from the exterior curved wall 66. The second and third aperture protrusions 67 and 68 are configured to fit within the base teeth undercut section 31 and axially interfere with the underside of the tooth head 29 to enable axially attachment of the end cap 40 or 42 to the implant body 20 base section 15 or 25. The second and third aperture protrusions 67 and 68 are located adjacent the seating surface 41 and extend away from the exterior curved wall 66. This positioning locates the second and third aperture protrusion 67 and 68 such that they can fit under the base tooth head 29 and within the undercut section 31 when the end cap 40 or 42 is axially positioned and secured to the implant body 20, as best shown in FIG. 5.

When the end cap 40 or 42 is axially positioned on the base section 15 or 30, a corresponding base extension 30, via the tooth head 29, will interact with the second and third aperture protrusions 67 and 68 as the base extension 30 enters the variable locking aperture 65. As the end cap 40 or 42 axially travels towards the fully seated position in the variable locking aperture 65, the second and third locking aperture protrusions 67 and 68 slideably travel along the topside of the tooth head 29 towards the implant base section 15 or 25 and a secured or locking position. Simultaneously, the opposing first locking aperture protrusion 57 also slideably travels along the topside of its corresponding tooth head 29 towards the implant base section 15 or 25 towards a secured or locking position. The result of this axial movement of the end cap 40 or 42 towards the implant base section 15 or 25 is that an opposing upward force is imparted by the base teeth 30 via the tooth head 29.

As the end cap 40 or 42 continues to be axially moved towards the implant base section 15 or 25, and as it continue to experience the opposing upward base teeth force, the end cap's 40 or 42 physical composition and resilient or spring-like properties and variable aperture slot 63 enable the end cap 40 or 42 to expand, deflect or flex in a manner that tends to enlarge the aperture slot 63. The material or composition make up of the end cap 40 or 42, and the relative position and configurations of the variable aperture 65 and aperture slot 63 provide the end cap 40 or 42 with physical characteristics and properties such that in a preferred aspect, the end cap 40 or 42 is resilient, spring-like, flexible expandable during axial positioning of the end cap 40 or 42 onto the implant base section 15 or 25.

As the end cap 40 or 42 continues to expand, the aperture slot 63 continues to enlarge which in turn permits the end cap 40 or 42 to continue axially movement towards the implant base section 15 or 25. Continued end cap 40 or 42 axially movement towards the implant base section 15 or 25 permits continued simultaneous sliding travel of the first, second and third locking aperture protrusions 57, 67 and 68 along the topside of corresponding tooth heads 29 towards the implant base section 15 or 25 and towards a secured or locking position. When the first, second and third locking aperture protrusions 57, 67 and 68 travel past the edge of a corresponding tooth head 29, the end cap 40 or 42 and the aperture slot 63 will cease expanding since the opposing upward force imparted on the end cap 40 or 42 by the base teeth 30 via the tooth head 29 is no longer present. With the opposing upward force imparted on the end cap 40 or 42 by the corresponding tooth heads 29 now removed and with continued axial end cap 40 or 42 movement towards the implant base section 15 or 25, the end cap's 40 or 42 resilient or spring-like properties will force or bias the end cap 40 or 42 back in a contracting or collapsing direction tending to make the end cap 40 or 42 and aperture slot 63 smaller. The end cap 40 or 42 will tend to deflect back to an equilibrium or static position, for example as shown in FIGS. 3 and 8. The final position of the end cap 40 or 42 and aperture slot 63 in the secured or locking position may be the same or different position as the end cap's 40 or 42 equilibrium or static position.

At this point, the end cap 40 or 42 and aperture slot 63 begin to contract as the end cap 40 or 42 continues axial movement towards the implant base section 15 or 25. The first, second and third locking aperture protrusions 57, 67 and 68 will then begin to radially move inward towards a secured or locking position under the corresponding base tooth head 29 and within the undercut base tooth section 31, as best shown in FIG. 5. Once the end cap 40 or 42 is axially and fully seated on the exterior surface 16 of the implant base section 15 or 25, base extensions 30 are located in corresponding fixed or variable locking aperture 55 and 65.

In the above preferred aspect description, it is contemplated that the end cap 40 or 42 is axially positioned such that the variable locking aperture 65 and fixed locking aperture 55 simultaneously approach and received corresponding base teeth 30 in the axial placement of the end cap 40 or 42 in a secured or locking position. However, those of skill in the art will recognize that a non-simultaneous approach could instead be used. For example, the fixed locking aperture 55 positioned such that the first locking aperture protrusion 57 is placed in a partially secured or locking position under the corresponding base tooth head 29 and within the undercut base tooth section 31. The end cap 40 or 42 would then be angled relative to the implant base section 15 or 25. While the end cap 40 or 42 is held in place on the fixed locking aperture side 55, the end cap could then be pivoted downward to bring the variable locking aperture 65 into a secured or locking position when the second and third locking aperture protrusions 67 and 68 are downwardly positioned under a corresponding base tooth head 29 and within undercut base tooth section 31. Those of skill in the art will thus recognize that the resilient or spring-like properties of the end cap 40 or 42 permit a variety of approaches axial or otherwise to secure the end cap 40 or 42 to the implant base section 15 or 25.

Once in the secure or locked position, the variable locking aperture 65 is in complementary and mechanical communication with the underside of the corresponding base tooth 29 and undercut section 31 of the base tooth 30 and in an axially obstructive fit with the base tooth underside. Once the end cap 40 or 42 is seated in the variable locking aperture 65, the underside of the tooth head 29, in cooperation with the variable aperture 65, will obstruct and prevent axial travel or movement of the positioning teeth 30 inside the variable locking aperture 65. If an attempt is made to axially remove or impart axial travel on the end cap 40 or 42 so as to unlock the end cap 40 or 42, the first, second and third locking aperture protrusions 57, 67 and 68 will abut up against the underside of corresponding tooth heads 29 which will prevent axial travel of the end cap 40 or 41. Also, if an attempt is made to rotationally move or force the end cap 40 or 42 to rotationally travel on the implant base section 15 or 25, the variable locking aperture's 65 substantially parallel sidewalls 61 and 62 will prevent rotational movement or travel of the end cap 40 or 42 relative to the positioning teeth 30, as best shown in FIG. 4. Simultaneously, the fixed locking aperture's 55 substantially parallel sidewalls 51 and 52 will also prevent rotational movement or travel of the end cap 40 or 42 relative to its corresponding positioning tooth 30. And, as will be discussed below, the secure positioning aperture's 75 sidewalls 71 and 22 will also simultaneously prevent rotational movement or travel of the end cap 40 or 42 relative to its corresponding positioning teeth 30.

The end cap 40 or 42 has thus reached a secured or locked position, as best shown in FIGS. 2 and 4, on the implant body base section 15 or 25. At this point, as shown in FIGS. 4 and 5, the second and third locking aperture protrusions 67 and 68 are in complementary and mechanical communication with the underside of a corresponding tooth head 29 and undercut section 31 of the base tooth 30 below the tooth head 29. In the secured or locked position, the end cap 40 or 42 is in an engaged or locked position relative to the implant body 20. The second and third locking aperture protrusions 67 and 68, the underside of the tooth head 29 and the undercut section 31 are preferably and complementarily configured such that, at the engaged and locked position, e.g., as shown in FIGS. 2, 4 and 5, the underside of the tooth head 29 will prevent or interfere with axial movement of the second and third locking aperture protrusions 67 and 68 away from the base teeth 30, and thereby axial movement of the end cap 40 or 42 away from the positioning teeth 30 and base section 15 and 25. The mechanical communication between the second and third locking aperture protrusion 67 and 68, the undercut section 31, the underside of the tooth head 29 and substantially parallel sidewalls 61 and 62 can comprise a second securing or locking mechanism 60.

The holding strength of the second locking mechanism 60 between the positioning tooth 30 and variable locking aperture 65, via the second and third locking aperture protrusions 67 and 68, underside of the base tooth head 29, and the undercut section 31, may be augmented or controlled by the addition or use of a coating or adhesive substance between the second and third locking aperture protrusion 67 and 69, the base tooth head underside and the undercut section 31. For example, a coating, such a silicone, or an adhesives such as an epoxy, may be used to increase friction between the second and third aperture protrusion 67 and 68, the base tooth head underside and the undercut section 31. Those of skill in the art will recognize that other substances or friction control mechanisms may be used to augment or control the holding strength between the variable locking aperture 65 and the positioning teeth 30, such as roughened surfaces, dissimilar materials, and shape differences.

The complementary and mechanical communication between the second and third locking aperture protrusions 67 and 68, the base tooth head 29 underside and the undercut section 31 will prevent axial movement or travel of the end cap 40 or 42 away from the implant base section 15 or 25 along the implant axis 5. This is the case since the second and third locking aperture protrusions 67 and 68 are now positioned underneath and obstructed by the base extension head 29. An attempt to axially move or remove the end cap 40 or 42 away from the implant base section would result in the second and third locking aperture protrusions 67 and 68 bumping into and abutting the underside of the tooth head in the undercut sections 31. The tooth head 29 prevents axial movement of the end cap 40 or 42 away from the implant base section 15 or 25 along the implant axis 5 once the end cap 40 or 42 is in a secured or locked position with the base section 15 or 25. Once in a locked position, the variable locking aperture 65 also prevents rotationally movement the end cap 40 or 42 to rotationally travel on the implant base section 15 or 25. This is accomplished via the variable locking aperture's 65 substantially parallel sidewalls 61 and 62 into which the base teeth 30 will abut into if rotational movement or travel is attempted once in the locked position, as best shown in FIG. 4.

FIGS. 2-8 also show that the end cap 40 or 42 further comprises four position securing apertures or passages 75 that are adapted to receive corresponding base teeth or base extensions 30 extending from the implant body 20 base sections 15 or 25. In a preferred aspect, the position securing apertures or passages 75 will simultaneously operate in combination with the fixed locking aperture 55 and variable locking aperture 65 to enable the end cap 40 or 42 to be axially attached and secured to the implant body 20 when the end cap 40 or 42 is axially placed on the base teeth 30 of the implant body base sections 15 or 25. In the embodiment disclosed, the position securing apertures or passages 75 are adapted to prevent rotational movement of the end cap 40 or 42 relative to the implant base section 15 or 25. In the disclosed aspect, the end cap 40 or 42 has four position securing apertures or passages 75. Those of skill in the art will recognize that an end cap 40 or 42 could instead have more or less position securing apertures or passages 75 in combination with the fixed and variable locking apertures 55 and 65 as might be desired or needed by a surgeon, medical procedure or clinical application. For example, in another aspect, the end cap 40 or 42 could instead have two position securing apertures or passages 75, two fixed apertures 55 and two variable locking apertures 65. Further, the position securing apertures or passages 75, the fixed locking aperture 55 and variable locking aperture 65 may have the same or different shape, configuration and/or sizes.

As best shown in, shown in FIGS. 2-8, the position securing apertures or passages 75 preferably comprises a pair of sidewalls 71 and 72 bounded by an exterior curved wall 73 and an opposing interior open space 74. The position securing apertures or passages 75 interact and cooperatively engage with corresponding base teeth heads 29 to enable the end cap 40 or 42 to be axially mounted onto the implant body 20. The position securing apertures or passages 75 are sufficiently sized and configured to permit the base teeth heads 29 to enter the position securing apertures or passages 75 between the sidewalls 71 and 72 as the end cap 40 or 42 is axially mounted or positioned onto the implant body 20 base section 15 or 25. When the end cap 40 or 42 is axially positioned on the base section 15 or 30, corresponding base extensions 30, via the teeth heads 29, will enter the position securing apertures or passages 75. As the end cap 40 or 42 axially travels to the fully seated position in the fixed and variable locking apertures 55 and 65, as described previously, the implant base teeth 30 fully enter and are positioned in the position securing apertures or passages 75 between the sidewalls 71 and 72 when the end cap 40 or 42 reaches a secured or locking position on the implant base section 15 or 25. The mechanical communication between the position securing apertures or passages 75 side walls 71 and 72 and the tooth head 29 can comprise a third securing mechanism 70, as shown in FIGS. 2 and 4.

Once the end cap 40 or 42 is in the secure or locked position on the implant base section 15 or 25, the sidewalls 71 and 72 will obstruct and prevent rotational travel or movement of the end cap 40 or 42 relative to the positioning teeth 30 which are now inside the position securing apertures or passages 75. If an attempt is made to rotationally move or force the end cap 40 or 42 to rotationally travel on the implant base section 15 or 25, the position securing apertures or passages 75 side walls 71 and 72, in conjunction with the fixed and variable locking apertures' 55 and 65 side walls 51, 52, 61 and 62, will prevent rotational movement or travel of the end cap 40 or 42 relative to the positioning teeth 30, as best shown in FIG. 4. The number of position securing apertures or passages 75 may vary from a single to multiple position securing apertures or passages 75. FIGS. 2-8 illustrate an embodiment with four (4) position securing apertures or passages 75. Those of skill in the art will recognize that more or less position securing apertures or passages 75 could instead be used as might be desired or needed by a surgeon, medical procedure or clinical application.

The end cap 40 or 42 will remain in the locked position until sufficient force is applied to overcome the first, second and third locking mechanisms 50, 60 and 70. Such force may be applied manually by a surgeon with the use and assistance of instruments (not shown). When such a removal force is introduced to the end cap 40 or 42, the first, second and third aperture protrusion 57, 67 and 68 will disengage the base teeth 30 which will in turn simultaneously release or disengage the first and second securing or locking mechanism 50 and 60. The end cap 40 or 42 can then be axially moved away from the base teeth 30 and implant base section 15 or 25.

FIGS. 2-8 show one preferred aspect of the end cap 40 or 42 of the present disclosure as discussed above. In this aspect, the end cap 40 or 42 comprises an end cap with six locking or securing apertures 55, 65 and 75. The end cap 40 or 42 comprises four position securing apertures or passages 75 and two locking apertures 55 and 65. The two locking apertures include a fixed locking aperture 55 with a first locking aperture protrusion 57 and a variable locking aperture 65 with corresponding second and third locking aperture protrusions 67 and 68 and a variable aperture slot 63. The variable aperture 63 enables the end cap 40 or 42 to resiliently deflect to expand and then contract as the end cap 40 or 42 is axially positioned on the implant base section 15 or 25. In this aspect, the locking apertures are preferably at least two opposing locking apertures 55 and 65 with corresponding locking aperture protrusions 57, 67 and 68, as best shown in FIGS. 4, and 6-8. In the preferred aspect, the variable locking apertures 65 comprises a variable aperture slot 63. Those of skill in the art will recognize that an end cap 40 can instead have other locking aperture arrangements, other opposing locking aperture pair orientations, more than two locking apertures, or a variable aperture slot which is not formed or positioned in a locking or positioning aperture. The specific configuration and arrangement of the end cap's locking apertures and/or variable aperture slot will depend on the desire or need of a surgeon, medical procedure or clinical application.

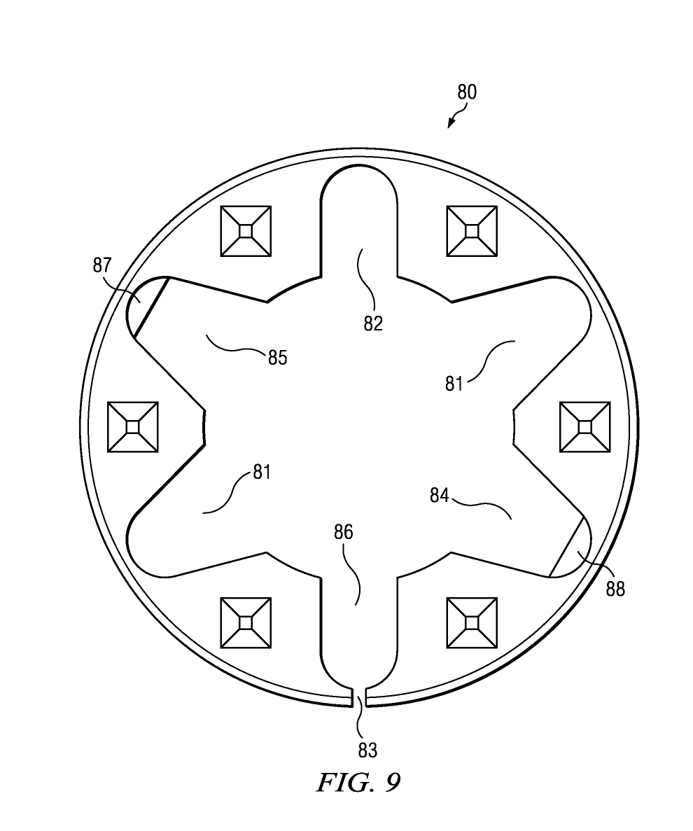

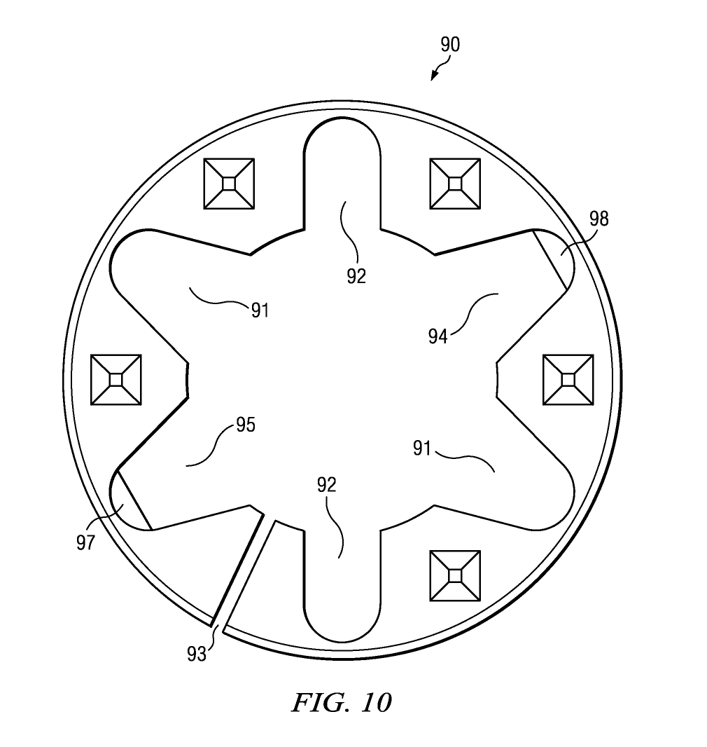

FIG. 9 shows a top view of an implant end cap 80 according to a second embodiment of the present disclosure. In this aspect, the end cap 80 comprises an end cap with six locking or securing apertures 81, 82, 84, 85 and 86. The end cap 80 comprises four position securing apertures 81, 82 and 86 and a pair of opposing locking apertures 84 and 85. The pair of opposing locking apertures 84 and 85 include corresponding locking protrusions 87 and 88. In this end cap 80 aspect, the variable aperture slot 83 is not formed as part of a locking aperture 84 or 85, but is instead formed as part of one of the positioning apertures 86. In this embodiment, the position securing apertures 84 and 85 via the locking protrusions 87 and 88 will engage corresponding base teeth 30 to enable axially securing of the end cap 80 to the implant base section 15, in a substantially similar manner as discusses above with respect to FIGS. 2-8. Further, the variable aperture 83 will enable the end cap 80 to resiliently deflect to expand and then contract as the end cap 80 is axially positioned on the implant base section 15 or 25.

FIG. 10 shows a top view of an implant end cap 90 according to a third embodiment of the present disclosure. In this aspect, the end cap 90 comprises an end cap with six locking or securing apertures 91, 92, 84 and 85. The end cap 90 comprises four position securing apertures 91 and 92 and two opposing locking apertures 94 and 95. The pair of opposing locking apertures 94 and 95 have a corresponding locking protrusion 97 and 98. FIGS. 8-10 also illustrate that the pair of opposing locking apertures 55 and 65, 84 and 85 and 94 and 95 can take on any one of three end cap orientations when there are six locking apertures. Further, FIG. 10 shows an end cap 90 with a variable aperture slot 93 that is not formed in any one of the locking apertures 91, 92 84 or 85. The variable aperture slot 93 is instead formed or configured as part of the end cap body. In this case, the variable aperture slot 93 is formed between the upper and lower surfaces of the end cap 90 and the external end cap wall and end cap interior. In this embodiment, the position securing apertures 94 and 95 via the locking protrusions 97 and 98 engage corresponding base teeth 30 to enable axially securing of the end cap 90 to the implant base section 15, in a similar manner as discusses above. The variable aperture 93 will enable the end cap 80 to resiliently deflect to expand and then contract as the end cap 90 is axially positioned on the implant base section 15 or 25.

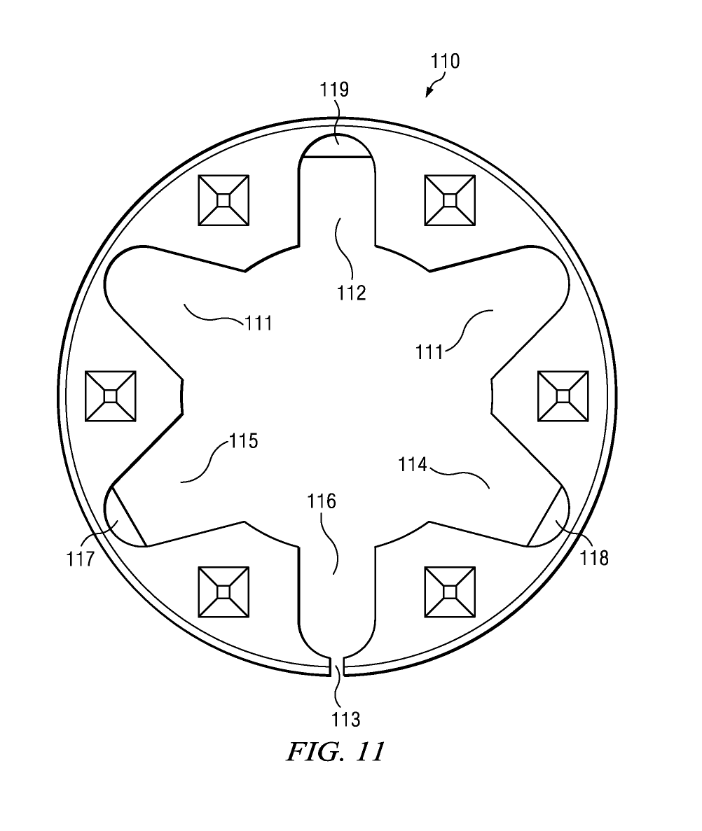

FIG. 11 shows a top view of an implant end cap 110 according to a fourth embodiment of the present disclosure. In this aspect, the end cap 110 comprises an end cap with six locking or securing apertures 111, 112, 114, 115 and 116. The end cap 110 comprises three position securing apertures 111 and 116 and three equidistantly spaced locking apertures 112, 114 and 115. This aspect shows that the end cap 110 may also have more than two locking apertures with corresponding locking protrusions 117, 118 and 119. An end cap will preferably have more than one locking aperture with locking protrusion. Further, in this aspect, the three locking apertures 112, 115 and 116 can take on any one of two end cap orientations since there are six locking apertures. In this end cap 110, the variable aperture slot 113 is not formed as part of a locking aperture 112, 114 or 115, but is instead formed as part of one of the positioning apertures 116. The variable aperture slot 113, however, could instead be formed or configured as part of the end cap body (not shown), for example similar to variable aperture slot 93 shown in FIG. 10. In this embodiment, the securing apertures 112, 114 and 115, via the locking protrusions 117, 118 and 119, engage corresponding base teeth 30 to enable axially securing of the end cap 110 to the implant base section 15 in a similar manner as discusses above. The variable aperture 113 will enable the end cap 110 to resiliently deflect to expand and then contract as the end cap 90 is axially positioned on the implant base section 15 or 25. Those of skill in the art will recognize other end cap configurations could also be used with the same or different number of locking or positioning apertures, and/or selective location and placement of a variable aperture slot as may be desired or needed by a surgeon, medical procedure or clinical application.

Referring back to FIGS. 2-8 show end caps 40 or 42 which have the same total number of end cap apertures 55, 65 and 75 as corresponding base teeth 30. The disclosed embodiment includes six total apertures 55, 65 and 75 and six corresponding base teeth or base extensions 30. If the end cap apertures or passages 55, 65 and 75 are to axially accept entry of extending base teeth 30, the end cap 40 must have at least the same number of end cap apertures 55, 65 and 75 as the number of extending base teeth 30. If there are two extending base teeth, then the there must be at least two apertures 55, 65 or 75. If there are four extending base teeth 30, then there must be at least four end cap apertures or passages 55, 65 and 75 in order that the end cap 40 or 42 can be axially inserted and seated onto the implant base section 15 or 25. Those of skill in the art will recognize that other embodiments may include an end cap 40 or 42 having more end cap apertures 55, 65 and 75 than corresponding base teeth 30. These embodiments would then include one or more empty end cap apertures 55, 65 and 75. Further, in other end cap aspects, the number of fixed locking apertures 55 the number of variable locking apertures 65 and the number of positioning apertures 75 could be the same or different.

An additional advantageous aspect of the disclosed end cap apertures or passages 55, 65 and 75 is that they enable the end cap 40 or 42 to be selectively positioned or adjusted on the implant base section 15 or 25. For example, during preassembly of the implant body 20 and end cap 40 or 42. The end cap 40 or 42 can be adjustable relative to the implant body 20 and implant base section 15 or 25 about the longitudinal axis 5 of the implant body 20 to determine a selected axial delivery position or orientation. The number of end cap apertures 55, 65 and 75 determine the number of positions or rotational orientations at which the end cap 40 or 42 can be axially placed in or located on the implant base section 15 and 25. The greater the number of end cap apertures 55, 65 and 75, the larger the number of positions or rotational positions the end cap 40 can be adjusted and axially placed in or located on the implant base section 15 and 25. The more end cap apertures or passages 55, 65 and 75, the greater degree of choice and control a surgeon will have in selecting a rotational position for the end cap 40 to be axially placed in or located on the implant base section 15 and 25. This end cap aspect advantageously provides a surgeon selective control of where the end cap angulation θ and the end cap vertex height H will be positioned on the implant base section 15 or 25. The ability to selectively position the end cap angulation θ permits a surgeon to determine where the end cap angulation θ and end cap height H will be applied or imparted to an the adjacent vertebral body 100 or 105. Prior to insertion of the implant 10 into the intervertebral disc space 101, the surgeon can decide where the end cap angulation θ and the end cap vertex height H are desired or needed for a particular medical procedure or clinical application.

As noted previously, a surgeon can selectively position the end cap 40 on the implant base plate 15 or 25 by rotating the end cap 40 relative to the base section 15 or 25, either clockwise or counterclockwise, and then axially inserting the end cap locking and positioning apertures 55, 65 and 75 onto the base teeth 30 at the desired or needed rotational position on the implant base plate 15. This aspect enables selective positioning or orientation of the end cap angulation θ which in turn permits the surgeon to decide where the end cap angulation θ and end cap height H will be applied or imparted to an the adjacent vertebral body 100 or 105. The clockwise or counterclockwise rotation of the end cap 40 or 42 moves or adjusts the end cap's 40 angulation θ and the end cap vertex height H relative to the implant base section 15 so as to position the end cap angulation θ and vertex height H at a desired or required point on the implant base section 15 or 25. For example at anterior, antereolateral, posterior or lateral points about the vertebral member 100 or 105, or vertebral disk space 101. This is in turn will position the end cap angulation θ and vertex height H at a desired or required point relative to the adjacent intervetebral member 100 or 105 once the implant 10 is inserted and positioned within the intervertebral space 101. The end cap 40 will then be able to impart desired or required angulation θ, orientation and vertex height H on the adjacent vertebral body at selected or required points on the adjacent vertebral body 100 or 105 to correct or improve the angulation, orientation, alignment and stabilization of the spine or spinal anatomy.

As noted above, the end cap 40 may be rotated so as to contact and impart angulation θ at different location points about the periphery of the adjacent vertebral body 100 or 105. The number of locking and positioning apertures or passages 55, 65 and 75 impact the incremental degree of control, through clockwise or counterclockwise end cap 40 rotation, that a surgeon will have in selecting the end cap angulation θ position between the implant 10 and the adjacent vertebral body 100 or 105. In the embodiment shown in FIGS. 2-8, the end cap 40 has six locking and positioning apertures 55, 65 and 75 which are evenly or equidistantly space in the area between the exterior contact surface 48 and seating surface 41. The equidistant spacing results in the locking and positioning apertures 55, 65 and 75 being located and spaced apart from each other at about sixty degrees (60°) around the end cap 40. In this embodiment then, the end cap 40 can be rotationally advanced, clockwise or counterclockwise, in single or multiple increments of sixty degrees (60°) in order to rotationally position or reposition the end cap angulation θ position between the implant 10 and the adjacent vertebral body 100 or 105.

A greater degree of control in rotationally and incrementally advancing the end cap 40, about the implant base section 15, may be obtained by increasing the number of locking and positioning apertures or passages 55, 65 and 75. For example, if the end cap 40 were to have eight (8) locking or positioning apertures or passages 55, 65 and 75 evenly or equidistantly spaced in the area between the contact surface 48 and seating surface 41. Then, equidistant circular spacing would result in the locking or positioning apertures or passages 55, 65 and 75 being located and spaced apart from each other at forty-five degrees (45°) around the substantially circular area between the contact surface 48 and seating surface 41 of the end cap 40. In this case, the end cap 40 can be rotationally advanced, clockwise or counterclockwise, in single or multiple increments of forty-five degrees (45°) in order to position or reposition the end cap angulation θ position between the implant 10 and the adjacent vertebral body 100 or 105. The larger number of locking apertures or passages 45 provides a surgeon the ability to rotationally position or reposition the end cap 40 in smaller discrete increments. This greater degree of control provides the surgeon with more precise control on where the end cap angulation θ will be positioned between the implant 10 and the adjacent vertebral body 100 or 105. In this manner, the selected angulation θ and end cap vertex height H can be imparted to an adjacent vertebral member 100 or 105 to thereby impart or drive angular orientation and height adjustment of the adjacent vertebral member 100 or 105 for correction or improved alignment, angulation, orientation, and stabilization of the spine or spinal anatomy.

In one aspect, assembling the implant 10 includes initially determining the type of end cap 40 or 42 that is to be attached to the body 20. The end cap 40 or 42 may be selected based on the size of the intervertebral space 101 and the anatomy of the vertebral members 100 and 105. The appropriate or desired axial approach position of the end cap 40 or 42 is then selected by a surgeon so that the end cap 40 or 42 can be axially placed on the on the implant base plate 15 or 25.

The proper end cap 40 or 42 and desired axial approach are determined, and the end cap 40 or 42 is axially placed on the base section 15 or 25 of the implant body 20. The one or more end cap apertures or passages 55, 65 and 75 are aligned with the one or more corresponding base teeth or base extensions 30 that axially extend outward from the base section support surface 16 of the implant body 20. The end cap 40 or 42 is axially moved towards the implant body 20 with the base teeth 30 to insert the base teeth 30 into the end cap apertures 55, 65 and 75. As the end cap 40 or 42 is axially positioned on the base section 15 or 30, a corresponding base extension 30, via the tooth head 29, will interact with the first locking aperture protrusions 57 as the base extension 30 enters the fixed locking aperture 55. Simultaneously, a corresponding base extension 30, via a corresponding tooth head 29, will interact with the second and third aperture protrusions 67 and 68 as the base extension 30 enters the variable locking aperture 65. Additionally, corresponding base extensions 30 will enter the position securing apertures 75.

As the end cap 40 or 42 continues to axially travel towards a locking position on the base section 15 or 25 of the implant body 20, first, second and third locking aperture protrusions 57, 67 and 68 slideably travel along the topside of a respective tooth head 29 towards the implant base section 15 or 25 and a secured or locking position. As the end cap 40 or 42 continues to axially move towards the implant base section 15 or 25, it experiences an opposing upward base tooth force which forces the end cap 40 or 42 to expand and deflect such that the aperture slot 63 is enlarged. As the end cap 40 or 42 continues its axial travel, the aperture slot 63 continues to enlarge which in turn permits the end cap 40 or 42 to continue travel the implant base section 15 or 25. When the first, second and third locking aperture protrusions 57, 67 and 68 travel past the edge of the corresponding tooth heads 29, the end cap 40 or 42 and the aperture slot 63 will cease expanding since the opposing upward tooth force, imparted on the end cap 40 or 42 base the tooth head 29, is no longer present.

With the opposing upward force imparted now removed, the end cap's 40 or 42 resilient or spring-like properties will force or bias the end cap 40 or 42 back in a contracting direction tending to make the aperture slot 63 smaller. The end cap 40 or 42 and aperture slot 63 contract which will permit the end cap 40 or 42 to continue axially movement towards the implant base section 15 or 25. The end cap 40 or 42 will deflect back towards an equilibrium or static position, as shown in FIGS. 3 and 8. The first, second and third locking aperture protrusions 57, 67 and 68 will then radially move inward towards a secured or locking position under the corresponding base tooth head 29 and within the undercut base tooth section 31, as best shown in FIG. 5. The final position or secured or locking position which may be the same or different position as the end cap's 40 or 42 equilibrium or static position. Once the end cap 40 or 42 seating surface 41 is fully seated on the exterior surface 16 of the implant base section 15 or 25, corresponding base teeth 30 are located in respective fixed and variable locking apertures 55 and 65 and position securing aperture 75. At this point, the end cap 40 or 42 is attached and secured to the implant body 20 base section 15 or 25 in an engaged or locked position.

Once in the engaged or locked position, shown in FIGS. 2, 4 and 5, the first, second and third locking aperture protrusions 57, 67 and 68 are in complementary and mechanical communication with the underside of corresponding tooth heads 29 and undercut section 31 of the base teeth 30 below the tooth heads 29. The first, second and third locking aperture protrusions 57, 67 and 68 and the corresponding underside of the tooth heads 29 will prevent axial movement of the first, second and third locking aperture protrusions 57, 67 and 68 away from the base teeth 30, and thereby prevent any axial movement of the end cap 40 or 42 away from the implant base section 15 and 25. Additionally, the apertures sidewalls 51, 52, 61 62, 71 and 72, of the fixed, variable and positioning passages 55, 65 and 75 respectively, will obstruct and prevent rotationally travel or movement of the end cap 40 or 42 relative to the implant base section 15 or 25.

The implants 10 and end caps 40, 42 may be implanted within a living patient for the treatment of various spinal disorders. The implants 10 and end caps 40, 42 may also be implanted in a non-living situation, such as within a cadaver, model, and the like. The non-living situation may be for one or more of testing, training, and demonstration purposes.

The end caps disclosed in this disclosure are preferably comprised of biocompatible materials substrates which can be used in combination with implants or devices configured to be inserted into an intervertebral space and contact against adjacent vertebral members. The biocompatible material substrate may include, among others, polyetheretherketone (PEEK) polymer material, homopolymers, co-polymers and oligomers of polyhydroxy acids, polyesters, polyorthoesters, polyanhydrides, polydioxanone, polydioxanediones, polyesteramides, polyaminoacids, polyamides, polycarbonates, polylactide, polyglycolide, tyrosine-derived polycarbonate, polyanhydride, polyorthoester, polyphosphazene, polyethylene, polyester, polyvinyl alcohol, polyacrylonitrile, polyamide, polytetrafluorethylene, poly-paraphenylene terephthalamide, polyetherketoneketone (PEKK); polyaryletherketones (PAEK), cellulose, carbon fiber reinforced composite, and mixtures thereof. The biocompatible material substrate may also be a metallic material and may include, among others, stainless steel, titanium, nitinol, platinum, tungsten, silver, palladium, cobalt chrome alloys, shape memory nitinol and mixtures thereof. The biocompatible material used can depend on the patient's need and physician requirements.

Spatially relative terms such as “under”, “below”, “lower”, “over”, “upper”, and the like, are used for ease of description to explain the positioning of one element relative to a second element. These terms are intended to encompass different orientations of the device in addition to different orientations than those depicted in the figures. Further, terms such as “first”, “second”, and the like, are also used to describe various elements, regions, sections, etc and are also not intended to be limiting. Like terms refer to like elements throughout the description.

As used herein, the terms “having”, “containing”, “including”, “comprising” and the like are open ended terms that indicate the presence of stated elements or features, but do not preclude additional elements or features. The articles “a”, “an” and “the” are intended to include the plural as well as the singular, unless the context clearly indicates otherwise.

While embodiments of the invention have been illustrated and described in the present disclosure, the disclosure is to be considered as illustrative and not restrictive in character. The present invention may be carried out in other specific ways than those herein set forth without departing from the scope and essential characteristics of the invention. The present embodiments are, therefore, to be considered in all respects as illustrative and not restrictive, and all changes coming within the meaning and equivalency range of the appended claims are intended to be embraced therein.

What is claimed is:

1. An implant for insertion into an intervertebral space between a first and second vertebral member, the implant comprising:
an implant body comprising a base section, the base section comprising a plurality of base extensions;
an end cap adapted for selective positioning at a selected point on the base section, the end cap comprising
an exterior contact surface that faces away from the implant body when the end cap is positioned on the base section,
a seating surface adapted to contact the base section when the end cap is positioned on the implant body,
an end cap angulation,
at least two locking apertures adapted to receive and engage corresponding base extensions, and
at least one positioning aperture adapted to receive a base extension,
a variable aperture slot adapted to enable the end cap to resiliently deform as the end cap is positioned on the base section;
wherein the locking apertures and positioning aperture are configured to complementarily engage corresponding base extensions to securely maintain the end cap positioned on the base section; and
wherein the implant imparts the end cap angulation to an adjacent vertebral body at the selected point when the implant is positioned in the intervertebral space.

2. The implant of claim 1, wherein the at least two locking apertures are opposing locking apertures.

3. The implant of claim 1, wherein the variable aperture slot is disposed in a locking aperture or a positioning aperture.

4. The implant of claim 1, wherein the end cap is axially positioned on the base section after rotational adjustment of the end cap about an implant axis.

5. The implant of claim 1, wherein the locking apertures and positioning aperture prevent rotational movement of the end cap relative to the base section.

6. The implant of claim 1, wherein the end cap angulation comprises an angular value in the range of between zero degrees to fifteen degrees (0°-15°).

7. The implant of claim 1, wherein the end cap angulation is an angular value selected from the group consisting of 0°, 4°, 8° and 15°.

8. The implant of claim 1, wherein the end cap further comprises an end cap height measured relative to the seating surface which enables the implant to both impart end cap height and end cap angulation to the adjacent vertebral body at the selected point.

9. The implant of claim 1
wherein the locking aperture and positioning aperture are spaced and located equidistantly about the end cap; and
wherein the plurality of base extensions are spaced and located equidistantly about the end cap.

10. An implant for insertion into an intervertebral space between a first and second vertebral member, the implant comprising:
an implant body comprising a base section, the base section comprising a plurality of base extensions;
an end cap adapted for selective positioning at a selected point on the base section, the end cap comprising
an exterior contact surface that faces away from the implant body when the end cap is positioned on the base section,
a seating surface adapted to contact the base section when the end cap is positioned on the implant body,
an end cap angulation,
a fixed aperture adapted to receive and engage a first base extension, and
a variable aperture adapted to receive and engage a second base extension,
wherein the variable aperture enables the end cap to resiliently deform as the end cap is positioned on the base section;
wherein the fixed aperture and variable aperture are configured to complementarily engage corresponding first and second base extensions to securely maintain the end cap positioned on the base section; and
wherein the implant imparts the end cap angulation to an adjacent vertebral body at the selected point when the implant is positioned in the intervertebral space.

11. The implant of claim 10, wherein the end cap further comprises at least one position securing aperture.

12. The implant of claim 10, wherein the end cap is axially positioned on the base section after rotational adjustment of the end cap about an implant axis.

13. The implant of claim 10, wherein the end cap angulation comprises an angular value in the range of between zero degrees to fifteen degrees (0°-15°).

14. The implant of claim 10, wherein the end cap further comprises an end cap height measured relative to the seating surface which enables the implant to both impart end cap height and end cap angulation to the adjacent vertebral body at the selected point.

15. An end cap adapted for use with an implant having an implant body with a base section having a plurality of base extensions, the end cap comprising:
an exterior contact surface;
a seating surface;
a substantially vertical exterior cap wall extending between the exterior contact surface and the seating surface;
at least two locking apertures extending between the exterior contact surface and the seating surface and adapted to receive and engage corresponding base extensions;
at least one positioning aperture extending between the exterior contact surface and the seating surface and adapted to receive and engage a base extension;
a variable aperture slot extending between the exterior contact surface and the seating surface and adapted to enable the end cap to resiliently deform as the end cap is positioned on the base section;
wherein the locking aperture and positioning aperture are configured to simultaneously and complementarily engage corresponding base extensions to securely maintain the end cap positioned on the base section;
wherein the locking apertures and positioning apertures enable the end cap to be axially positioned at a selected point on the implant base section; and
wherein the end cap positioned on the implant imparts an end cap angulation to an adjacent vertebral body at the selected point when the implant is positioned in an intervertebral space.

16. The implant of claim 15, wherein the at least two locking apertures are opposing locking apertures.

17. The implant of claim 15, wherein the variable aperture slot is disposed in a locking aperture or a positioning aperture.

18. The end cap of claim 19, wherein the end cap is selectively and axially positionable on the base section via rotational adjustment of the end cap about an implant longitudinal axis so that the end cap angulation coincides to the selected point.

19. The end cap of claim 15, wherein the end cap angulation comprises an angular value in the range of between zero degrees to fifteen degrees (0°-15°).

20. A method of assembling an implant for insertion into an intervertebral space between a first and second vertebral member, the method comprising:
positioning an end cap at an end of an implant body, the end cap having a fixed aperture adapted to receive a corresponding first base extension that extends from the implant body, a variable aperture adapted to receive a corresponding second base extension that extends from the implant body, and a least one position securing apertures adapted to receive corresponding base extensions that extend from the implant body;
axially inserting the first and second base extensions into a corresponding end cap fixed aperture and variable aperture, and at least one base extension into the at least one position securing apertures;
resiliently deforming the end cap via a variable aperture slot during axial insertion of the end cap onto the implant body to enable the first, second and third locking protrusions to lockingly engage the first and second base extensions in the fixed and variable apertures, and to permit base extensions to be received in corresponding position securing apertures; and
securing the first and second base extensions within the corresponding fixed and variable apertures, and positioning the base extensions into the at least one position securing apertures to thereby position the end cap to the implant body in a locked position.
